(19) United States
(12) Patent Application Publication
(10) Pub. No.: US 2013/0303628 A1
(43) Pub. Date: Nov. 14, 2013

(54) CURCUMINOID SOLID DISPERSION FORMULATION

(71) Applicants: Jörg Breitenbach, Mannheim (DE); Thomas K. Kessler, Schifferstadt (DE); Katrin Schneider, Mannheim (DE); Tapas Das, Worthington, OH (US); Shreeram Sathyavageeswaran, Singapore (SG); Ai Mey Chuah, Singapore (SG); Guarav C. Patel, Gahanna, OH (US)

(72) Inventors: Jörg Breitenbach, Mannheim (DE); Thomas K. Kessler, Schifferstadt (DE); Katrin Schneider, Mannheim (DE); Tapas Das, Worthington, OH (US); Shreeram Sathyavageeswaran, Singapore (SG); Ai Mey Chuah, Singapore (SG); Guarav C. Patel, Gahanna, OH (US)

(73) Assignees: ABBOTT LABORATORIES, Abbott Park, IL (US); ABBOTT GMBH &I CO. KG, Wiesbaden (DE)

(21) Appl. No.: 13/878,731

(22) PCT Filed: Oct. 13, 2011
(86) PCT No.: PCT/EP2011/067901

§ 371 Date: Jul. 17, 2013

Related U.S. Application Data
(60) Provisional application No. 61/393,206, filed on Oct. 14, 2010.

(51) Int. Cl. A61K 31/12

(52) U.S. Cl. CPC A61K 31/12
USPC 514/679

(57) ABSTRACT

A curcuminoid formulation, comprising a melt-processed solid dispersion product comprising one or more curcuminoids, a nutritionally acceptable thermoplastic polymer, and a phosphatide; providing an improved oral bioavailability compared to non-formulated crystalline curcuminoid. A method for producing said formulation. A nutritional product fortified with said formulation. Said formulation for use in the treatment or prophylaxis of cancer, conditions involving an inflammatory reaction, neurological disorders, cardiovascular disease, pulmonary disease, the formation of cholesterol gallstones, and parasitic infestation.

### FIELD OF THE INVENTION

Curcumin is the principal curcuminoid of the popular Indian spice turmeric, which is a member of the ginger family (Zingiberaceae). Other naturally occurring curcuminoids are desmethoxycurcumin and bis-desmethoxycurcumin. Curcuminoids are polyphenols and are responsible for the yellow color of turmeric. Curcuminoids can exist in at least two tautomeric forms, keto and enol. The enol form is more energetically stable in the solid phase and in solution. Curcumin is practically insoluble in water at acidic and neutral pH; and rapidly decomposes at alkaline pH. Curcumin is stable under dry conditions and also relatively stable to heat.

Curcumin is a very powerful antioxidant. Its antioxidant effect has been reported to be eight times more powerful than that of vitamin E. A number of studies provide evidence for the therapeutic properties of naturally occurring curcuminoids and synthetic curcuminoid derivatives, in particular their anti-cancer activity (for example, Pisano et al., Mol. Cancer. 2010, 3;9(1):137; Bisht et al., J. Nanobiotechnology. 2007, 5:3). There are also reports on other pharmacological activities of curcumin including anti-microbial and anti-inflammatory effects (Begum et al., J Pharmacol Exp Ther. 2008, 326(1):196-208).

Although curcuminoids have been suggested for a variety of therapeutic and prophylactic applications, a major impediment in this development is the very low bioavailability of orally administered curcumin. For example, it has been reported that serum levels in humans after an oral dose of 2 g curcumin alone were either undetectable or very low. Reasons contributing to this effect are the low stability and poor absorption of curcumin in the digestive tract as well as its rapid metabolism, in particular in the liver, and rapid systemic elimination. Thus, the serum curcumin levels sufficient to provoke the desired beneficial effect of this compound cannot be achieved by the mere consumption of turmeric with the food. Concomitant administration of piperine, an inhibitor of enzymes involved in drug metabolism, has been shown to increase the curcumin absorption and thus the serum concentration of curcumin. However, it is a significant downside of this approach that the piperine induced inhibition of drug metabolism may lead to unwanted effects, in particular when other medications are taken. (Anand et al., Mol Pharm 2007 4(6):807-18; Shoba et al., Planta Med 1998 64(4):353-6)

Different formulations of curcumin have been developed to circumvent the poor aqueous solubility and/or oral bioavailability of curcumin. In 2007, a polymeric nanoparticle-encapsulated formulation of curcumin (“nanocurcumin”) with less than 100 nm particle size has been reported by Bisht et al. (J. Nanobiotechnology. 2007, 5:3) to be readily dispersable in aqueous media and to show comparable efficacy compared to free curcumin in human cancer cell line models. A “lipidated” curcumin formulation has been described by Begum et al. (J Pharmacol Exp Ther. 2008, 326(1):196-208) to result in 11-fold higher plasma curcumin levels and 4-fold higher levels in brain compared with equal doses of curcumin powder or curcumin-piperine extracts after oral administration. In WO2010/010431 liquid and semisolid self-emulsifying curcumin formulations based on a lipid carrier system of PEG fatty acid esters are described which showed improved bioavailability compared to an aqueous suspension of curcumin after oral administration to rats. Curcumin nanoparticles and curcumin bound to chitosan nanoparticles were described to provide improved oral bioavailability of curcumin in mice compared to curcumin orally administered in olive oil (WO 2010/013224). Paradkar et al. (Int J. Pharm. 2004, 271(1-2):281-6) describe curcumin-PVP solid dispersions obtained by spray drying that showed increased dissolution rates compared to pure curcumin or physical curcumin mixtures. Curcumin formulations including a nanocrystal solid dispersion in hydroxypropyl cellulose, an amorphous solid dispersion in hydroxypropylmethyl cellulose and a nanoemulsion in a solvent mixture comprising PEG400 have been described by Onoue et al. (J Pharm Sci. 2010, 99(4):1871-81). Each of these three curcumin formulations has been reported to show improved solubility in water as well as higher plasma curcumin concentrations in rats after oral administration compared to pure crystalline curcumin.

Despite the various approaches described above there is still a tremendous need to provide curcumin formulations which allow optimum bioavailability of curcumin when administered orally.

### SUMMARY OF THE INVENTION

Thus, this invention provides a curcuminoid formulation, comprising a melt-processed solid dispersion product comprising
a) one or more curcuminoids,
b) a nutritionally acceptable thermoplastic polymer, and
c) a phosphatide.

The solid dispersion product comprises a matrix of the nutritionally acceptable thermoplastic polymer and phosphatide wherein the curcuminoid(s) are homogeneously distributed. Beside the solid dispersion product, the curcuminoid formulation may comprise one or more other ingredients, e.g., additives or other nutritionally desirable ingredients.

The invention further provides a method for producing the curcuminoid formulation described herein, comprising:
a) blending one or more curcuminoids, a nutritionally acceptable thermoplastic polymer, and a phosphatide;
b) heating the blend to obtain a homogeneous melt;
c) forcing the thus obtained melt through one or more nozzles;
d) allowing the melt to solidify to obtain a solid dispersion product.

The invention also provides a nutritional product fortified with a curcuminoid formulation as described herein as well as the curcuminoid formulation described herein for use in the treatment or prophylaxis of diseases and conditions such as cancer, conditions involving an inflammatory reaction, neurological disorders, cardiovascular disease, pulmonary disease, the formation of cholesterol gallstones, and parasitic infestation.

### BRIEF SUMMARY OF THE FIGURES

FIGS. 1A-J show graphs illustrating the curcuminoid concentrations in rat plasma after oral administration of curcuminoids. 4 to 5 rats were orally administered with an amount of curcuminoid formulation #1.1, #1.2, #2.1, #2.2, #3.1, #3.2, #3.3, #4.1 or #4.2 comprising 20 mg curcuminoids (FIGS. 1A-I). The control subjects were administered with 20 mg non-formulated crystalline curcuminoids (FIG. 1J).

### DETAILED DESCRIPTION OF THE INVENTION

The term “curcuminoid”, as used herein, refers to curcumin and derivatives thereof and analogs thereof. These include natural and synthetic derivatives of curcumin, and any combination of more than one curcuminoid.

In particular, for the purposes herein, the term “curcuminoid” should be understood to encompass compounds having a 1,7-bis(4-hydroxyphenyl)-1,6-heptadiene-3,5-dione or 1,7-bis(4-hydroxyphenyl)hept-4-en-3-one skeleton wherein the phenyl groups independently may bear one or more alkoxy residues, especially one methoxy residue in 3-position.

Naturally occurring curcuminoids comprise curcumin (1,7-Bis-(4-hydroxy-3-methoxyphenyl)-hepta-1,6-diene-3,5-dione), desmethoxycurcumin (1-(4-Hydroxyphenyl)-7-(4-hydroxy-3-methoxyphenyl)-hepta-1,6-diene-3,5-dione) and bis-desmethoxycurcumin (1,7-Bis-(4-hydroxyphenyl)-hepta-1,6-diene-3,5-dione). These compounds can exist in at least two tautomeric forms, keto and enol having the structural formulae

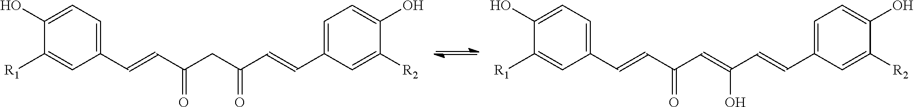

wherein in curcumin R₁ and R₂ are methoxy, in desmethoxycurcumin R₁ is hydrogen and R₂ is methoxy, and in bis-desmethoxycurcumin R₁ and R₂ are hydrogen.

Analogs of curcumin include those derivatives disclosed in U.S. Pat. No. 6,664,272 and Pisano et al., Mol. Cancer. 2010, 3; 9(1):137, which are herein specifically incorporated by reference.

The term “nutritionally acceptable”, as used herein, refers to a compound that does not cause acute toxicity when the curcumin formulation of the invention comprising it is ingested as a supplement for a nutritional product, or is administered orally in treatment or prophylaxis of diseases and conditions such as cancer, conditions involving an inflammatory reaction, neurological disorders, cardiovascular disease, pulmonary disease, the formation of cholesterol gallstones, and parasitic infestation. Expediently, all components of the curcumin formulation of the present invention are nutritionally acceptable.

The nutritionally acceptable thermoplastic polymer used in the solid dispersion product described herein is a polymer capable to act as a solid meltable solvent. It forms a matrix for dispersion, and in particular for dissolution, of the curcuminoid. Preferably, said polymer is at least partly soluble or swellable in aqueous media, expediently under the conditions of use, i.e. in particular under physiological conditions in the digestive tract. Most preferably, said polymer is a water-soluble polymer.

The nutritionally acceptable thermoplastic polymer may be selected from

homopolymers and copolymers of N-vinyl lactams, especially homopolymers and copolymers of N-vinyl pyrrolidone, e.g. polyvinylpyrrolidone (PVP), copolymers of N-vinyl pyrrolidone and vinyl acetate or vinyl propionate,

cellulose derivatives, such as cellulose esters, cellulose ethers and cellulose ether esters, in particular methylcellulose and ethylcellulose, hydroxyalkylcelluloses, in particular hydroxypropylcellulose, hydroxyalkylalkylcelluloses, in particular hydroxypropylmethylcellulose, cellulose phthalates or succinates, in particular cellulose acetate phthalate and hydroxypropylmethylcellulose phthalate, hydroxypropylmethylcellulose succinate or hydroxypropylmethylcellulose acetate succinate;

high molecular polyalkylene oxides such as polyethylene oxide and polypropylene oxide and copolymers of ethylene oxide and propylene oxide,

polyvinyl alcohol-polyethylene glycol-graft copolymers (available as Kollicoat® IR from BASF SE, Ludwigshafen, Germany);

graft copolymers comprising a poly(alkylene glycol) backbone and a vinyl acetate/N-vinylcaprolactam copolymer grafted onto the backbone (available as Soluplus® from BASF SE, Ludwigshafen, Germany);

polyacrylates and polymethacrylates such as methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), poly(hydroxyalkyl methacrylates),

polyacrylamides,

vinyl acetate polymers such as copolymers of vinyl acetate and crotonic acid, partially hydrolyzed polyvinyl acetate (also referred to as partially saponified “polyvinyl alcohol”),

polyvinyl alcohol,

oligo- and polysaccharides such as carrageenans, galactomannans and xanthan gum,

polyhydroxyalkanoates, e.g. polyhydroxybutyric acid, polylactic acid, and poly(lactide-co-glycolide);

polyamino acids, e.g. polylysine, polyasparagine,

proteins and polypeptides, such as gelatin

or mixtures of one or more thereof.

According to one aspect of the invention, the nutritionally acceptable thermoplastic polymer is selected from cellulose derivatives, especially hydroxyalkylcelluloses and hydroxyalkylalkylcelluloses.

Preferred water-soluble polymers are for example hydroxypropylmethylcellulose (Methocel, Pharmacoat), polymethacrylate (Eudragit EO), hydroxypropylcellulose (Klucel), or a polyvidone.

Especially preferred water-soluble polymers are hydroxypropylmethylcelluloses or HPMC (INN name: hypromellose). Said HPMC contains sufficient hydroxypropyl and methoxy groups to render it water-soluble. HPMC having a methoxy degree of substitution from about 0.8 to about 2.5 and a hydroxypropyl molar substitution from about 0.05 to about 3.0 are generally water-soluble. Methoxy degree of substitution refers to the average number of methyl ether groups present per anhydroglucose unit of the cellulose molecule. Hydroxypropyl molar substitution refers to the average number of moles of propylene oxide which have reacted with each anhydroglucose unit of the cellulose molecule. Preferably hydroxypropylmethylcellulose with low viscosity, i.e. about 5 mPa·s, is used, e.g. hydroxypropylmethylcellulose 2910 5 mPa·s. In the four digit number “2910”, the first two digits represent the approximate percentage of methoxyl groups and the third and fourth digits the approximate percentage composition of hydroxypropoxyl groups. 5 mPa·s is a value indicative of the apparent viscosity of a 2% aqueous solution at 20° C. Suitable HPMC include those having a viscosity from about 1 to about 100 mPa·s, in particular form about 3 to about 15 mPa·s, preferably about 5 mPa·s The most preferred type of HPMC having a viscosity of 5 mPa·s., is the commercially available HPMC 2910 5 mPa·s.

The term “phosphatide”, as used herein, refers to compounds which are derivatives of glycero-3 phosphoric acid that contains at least one O-acyl, O-alkyl or O-alk-1′-enyl residue attached to the glycerol moiety and a polar head made of a nitrogenous base, a glycerol, or an inositol unit. The terms “phosphatide”, “glycerophospholipid” and “phosphoglyceride” are used interchangeably. In a further particular embodiment of the invention the phosphatide comprised in the solid dispersion product described herein is a lecithin. Lecithins are in particular phosphatidylcholines, i.e. a group of phosphatides composed of phosphoric acid, choline, and fatty acids.

According to one aspect of the invention, the solid dispersion product additionally comprises a normally solid polyol, i.e. “normally solid” means that the polyol is solid at NTP (Normal Temperature and Pressure, i.e., 20° C. and 1 atm). The normally solid polyol acts as a melting point or softening point depressant and facilitates the uniform incorporation of the curcuminoid(s) into the solid dispersion matrix. Without wishing to be bound to theory, we believe that the normally solid polyol acts as a plasticizer for the nutritionally acceptable polymer and/or the normally solid polyol initially melts and the other component(s) will dissolve in the melt.

The normally solid polyol can be selected from, e.g., high molecular weight polethyllenglycols.

Preferably, however, said polyol is a sugar alcohol. A sugar alcohol is a hydrogenated form of carbohydrate, whose carbonyl group has been reduced to a primary or secondary hydroxyl group. Said sugar alcohol may be selected from maltitol, mannitol, sorbitol, lactitol, xylitol, erythritol and isomalt. In a particular embodiment of the invention the polyol is isomalt, also referred to as 1-O-alpha-D-glycopyranosyl-D-mannitol or Palatinit.

The solid dispersion product described herein may comprise
a) 0.1 to 50%, for example 5 to 30% or 10 to 20%, by weight of the curcuminoid(s),
b) 20 to 95%, for example 40 to 80%, by weight of the nutritionally acceptable thermoplastic polymer,
c) 5 to 50%, for example 5 to 25%, by weight of the phosphatide, and
d) 0 to 50%, for example 1 to 30% or 5 to 15%, by weight of the normally solid polyol.

Various additives may be included in the curcuminoid formulation of the invention, for example lubricants, fillers, disintegrants, preservatives or stabilizers such as antioxidants, light stabilizers, radical scavengers and stabilizers against microbial attack, dyes such as azo dyes, organic or inorganic pigments such as iron oxides or titanium dioxide, or dyes of natural origin, pH regulators, as well as compounds which alter or mask flavor and/or odor of the curcuminoid formulation such as sweeteners, flavorings and odorants.

At least part of the curcuminoid(s) is/are homogeneously dispersed in the matrix of the solid dispersion product. It is particulary preferred that the curcuminoid(s) in the solid dispersion product is/are present in an essentially non-crystalline state. This encompasses a state wherein essentially amorphous domains of curcuminoid(s) are interspersed in the matrix and a state wherein the curcuminoid(s) are molecularly dispersed in the matrix. When said dispersion of the components is such that the system is chemically and physically uniform or homogeneous throughout or consists of one phase (as defined in thermodynamics), such a solid dispersion will be called a “solid solution” or a “molecular dispersion”. The state of molecular dispersion corresponds to the maximum possible homogenization of the curcuminoid in the matrix.

Known analytical methods can be used to investigate the state of such solid dispersions, for example differential scanning calorimetry (DSC) or wide angle X-ray scattering measurements (WAXS measurements). The DSC analytical measurement of an essentially non-crystalline state lacks the melting peak which occurs with the crystalline pure substance and is usually endothermic. Another possibility for identifying an essentially non-crystalline state is the reduction in intensity and/or absence of typical X-ray diffraction signals in the WAXS analysis.

Stability of curcuminoids is particularly high under dry conditions. Therefore the curcumin formulations of the invention preferably comprise less than 5% per weight, for example less that 1% per weight, water. In a particular embodiment of the invention the curcumin formulation is essentially anhydrous.

The solid dispersion product can be produced by blending one or more curcuminoids, a nutritionally acceptable thermoplastic polymer, and a phosphatide, heating the blend to obtain a homogeneous melt, and allowing the melt to solidify to obtain a solid dispersion product. The terms “melt” and “melting” should be interpreted broadly. For the purposes herein, these terms not only mean the alteration from a solid state to a liquid state, but can also refer to a transition to a glassy state or a rubbery state, and in which it is possible for one component of the mixture to get embedded more or less homogeneously into the other. In particular cases, one component will melt and the other component(s) will dissolve in the melt, thus forming a solution, which, upon cooling, may form a solid solution having advantageous dissolution properties. Blending and heating are conveniently performed in a mixer or kneader which is jacketed for heating.

A preferred method for producing the curcuminoid formulation of present invention comprises:
a) blending one or more curcuminoids, a nutritionally acceptable thermoplastic polymer, and a phosphatide;
b) heating the blend to obtain a homogeneous melt;
c) forcing the thus obtained melt through one or more nozzles;
d) allowing the melt to solidify to obtain a solid dispersion product.

Steps a) to c) may be performed in one or more than one apparatus suitable for this purpose, such as an extruder or kneader extruder. Preferable the blend is subjected to a mixing action in a mixing section of the extruder.

Extruders are known per se. An extruder comprises a housing or barrel divided into several sections in a longitudinal direction. On the upstream side of the extruder, an opening is provided for feeding powders of the curcuminoid(s), the nutritionally acceptable thermoplastic polymer, the phosphatide and any further components such as the normally solid polyol and/or additives described above. Usually, a hopper is placed on this opening so that the powder can be easily fed into the barrel of the extruder. The barrel ends in conveying direction in a die, where the dispersion is expelled.

The extruder comprises at least one rotating shaft. Alternatively, it may comprise two or up to six rotating shafts. In preferred embodiments, the extruder is a twin-screw extruder. The shafts may be co-rotating or counter-rotating, but are preferably co-rotating. Processing elements disposed on adjacent shafts closely intermesh.

Each shaft carries a plurality of processing elements disposed axially one behind the other. The processing elements define a feeding and conveying section, at least one mixing section, and a discharging section. The feeding and conveying section is positioned farthest upstream, close to the hopper of the extruder, the at least one mixing section is positioned downstream of the feeding and conveying section, and the discharging section is positioned farthest downstream, close to the discharge opening of the extruder. The term “downstream” as used herein, refers to direction in which the material is being conveyed in the extruder, i.e. the conveying direction.

The processing elements of the feeding and conveying section as well as the discharging section are formed by screw-type elements. Preferably, these screw type elements form an endless screw having the feed direction and a uniform pitch flight. Thus, in the feeding and conveying section the powder is fed into the extruder and conveyed in the downstream direction, for example at a feed rate of 0.5 to 1.5 h, preferably of 0.5 to 1.0 kg/h.

In the mixing section(s) the material to be processed is homogenized by mixing or kneading. Suitably, paddle means or kneading blocks may be used. These kneading blocks consist of cam disks mutually offset at an angle in a peripheral direction. The cam disks have abutting faces that are perpendicular to the general conveying direction in the extruder. Alternatively, the mixing section(s) are defined by processing element(s) that comprise(s) a mixing element that is derived from a screw type element. A mixing element “being derived from a screw type element” is intended to mean an element whose basic shape is that of a screw element, but which has been modified such that it exerts a compounding or mixing effect in addition to a conveying effect.

In a preferred embodiment of the invention, the extruder comprises one or more than one, for example three or four, mixing sections, which are connected by intermediate conveying sections formed by screw-type elements.

The extruder shaft may further comprise one or more than one reverse-flight section(s), preferably arranged after the (last) mixing section and defined by reverse-flight elements. A reverse-flight element has a screw with a reverse-flight relative to the screw-type elements which may be arranged in the feeding and conveying section which define the general conveying direction of the extruder. Thus, the reverse-flight element convey the material in an opposite direction relative to the general conveying direction of the extruder and serves to create sufficient back-pressure to allow for a desired degree of mixing and/or homogenization. The reverse-flight element is designed to stow the material conveyed in the extruder. Therefore it may also be called a back-pressure element.

The substances which are fed to the extruder are melted in order to homogenize the melt and to disperse or, preferably, dissolve the curcuminoid in the matrix efficiently.

“Melting” means transition into a liquid or rubbery state in which it is possible for one component to be homogeneously embedded in the other. Melting usually involves heating above the softening point of the polymer. Usually, the maximum melt temperature is in the range of 90 to 180° C., preferably 100 to 160° C.

The extruder housing is heated in order to form a melt from the substances fed to the extruder. It will be appreciated that the working temperatures will also be determined by the kind of extruder or the kind of configuration within the extruder that is used. A part of the energy needed to melt, mix and dissolve the components in the extruder can be provided by heating elements, while the friction and shearing of the material in the extruder can also provide the mixture with a substantial amount of energy and aid in the formation of a homogeneous melt of the components.

In order to obtain a homogeneous distribution and a sufficient degree of dispersion of the active ingredient, the curcuminoid-containing melt is kept in the heated barrel of the melt extruder for a sufficient length of time.

According to one aspect of the invention, the barrel of the extruder is divided into several heating zones. The temperature in these heating zones can be controlled in order to control the melting of the dispersion. Preferably, the portion of the barrel upstream of the first mixing element or first reverse-flight element is maintained at a lower temperature, e.g. a temperature of about 50 to about 100° C., than the portion of the barrel downstream of the first mixing element that is kept at, e.g., about 100 to about 150° C.

The extrudate exiting from the extruder ranges from pasty to viscous. Before allowing the extrudate to solidify, the extrudate may be directly shaped into virtually any desired shape. Shaping of the extrudate may be conveniently carried out by a calender with two counter-rotating rollers with mutually matching depressions on their surface. A broad range of tablet forms can be attained by using rollers with different forms of depressions. If the rollers do not have depressions on their surface, films can be obtained. Alternatively, the extrudate is molded into the desired shape by injection-moulding. Alternatively, the extrudate is subjected to profile extrusion and cut into pieces, either before (hot-cut) or after solidification (cold-cut).

Optionally, the resulting solid dispersion product is milled or ground to granules. The granules may then be compacted. Compacting means a process whereby a powder mass comprising the granules is condensed under high pressure in order to obtain a compact with low porosity, e.g. a tablet. Compression of the powder mass is usually done in a tablet press, more specifically in a steel die between two moving punches.

A number of uses may be contemplated for the curcuminoid formulations of the present invention. In particular, the curcumin formulations of the invention can be used in the nutritional and pharmaceutical sector for both, humans and non-human animals. For example, said applications comprise the use of the curcumin formulations in the treatment or prophylaxis of diseases and conditions including cancer, in particular solid tumors such as colorectal, lung, breast, pancreatic and prostate carcinoma. Further potential pharmacological uses are in the treatment or prophylaxis of conditions involving an inflammatory reaction such as arthritis, neurological disorders such as Alzheimer's disease and Parkinson's disease, cardiovascular disease, pulmonary disease, the formation of cholesterol gallstones, and parasitic infestation such as by Plasmodium (e.g. malaria pathogen), Trypanosoma and Leishmania. For these purposes, the formulations of the invention may be administered alone, for example as dietary supplements, or together with further excipients, drugs or as food ingredients.

For oral administration of the formulations of the invention a variety of dosage forms may be used comprising liquid or semisolid forms such as emulsions, microemulsions and suspensions, and solid forms such as granules, capsules, pellets, powders or tablets.

Emulsions and microemulsions may be of the oil-in-water or water-in-oil type and contain formulations of the invention as disperse or dispersing phase. These emulsions or microemulsions may be stabilized by the presence of emulsifiers known to be used for this purpose.

Granules consist of solid grains of formulations of the invention, each grain representing an agglomerate of powder particles. A lubricant is preferably used in compacting the granules. Suitable lubricants are selected from polyethylene glycol (e.g., having an Mw of from 1000 to 6000), magnesium and calcium stearates, sodium stearyl fumarate, and the like. The user can be offered single-dose preparations, for example granules packed in a small bag (sachet), a paper bag or a small bottle, or multidose preparations which require appropriate dimensions. However, in many cases, such granules do not represent the actual drug form, but are intermediates in the manufacture of particular drug forms, for example tablet granules to be compressed to tablets, capsule granules to be packed into hard gelatin capsules, or instant granules or granules for oral suspension to be put in water before intake.

As capsules, the formulations of the invention are usually packed into a hard shell composed of two pieces fitted together or a soft, one-piece, closed shell, which may vary in shape and size. It is likewise possible for formulations of the invention to be encased or enveloped or embedded in a matrix in suitable polymers, i.e. microcapsules and microspherules. Hard and soft capsules consist mainly of gelatin, while the latter have a suitable content of plasticizing substances such as glycerol or sorbitol. Hard gelatin capsules are used to receive formulations of the invention which have a solid consistency, for example granules, powder or pellets. Soft gelatin capsules are particularly suitable for formulations with a semisolid consistency and, if required, also viscous liquid consistency.

Pellets are granules of formulations of the invention in the particle size range from about 0.5 to 2 mm in diameter. Both with a narrow particle size distribution, preferably from 0.8 to 1.2 mm, and with an essentially round shape, are preferred.

Tablets are solid preparations in particular for oral use. The meaning of “oral administration” within the framework of the present invention is, in particular, that of the term “peroral administration” or “ingestion”, thus the tablets are for absorption or action of the curcuminoid in the gastrointestinal tract. Particular embodiments are coated tablets, layered tablets, laminated tablets, tablets with modified release of the curcuminoid, matrix tablets, effervescent tablets, chewable tablets or pills. The formulations of the invention usually comprise at least a part of the necessary tablet excipients, such as binders, fillers, glidants and lubricants, and disintegrants. Tablets of formulations of the invention may also, if necessary, comprise other suitable excipients, for example excipients which assist tableting such as lubricants and glidants, e.g. magnesium, aluminum and calcium stearates, talc and silicones, animal or vegetable fats, especially in hydrogenated form and those which are solid at room temperature. Coated tablets additionally comprise suitable coating materials, for example film coating agents with coating aids, especially those mentioned below. Coated tablets include, in particular, sugar-coated tablets and film-coated tablets.

Powders are finely dispersed solids of formulations of the invention with particle sizes usually of less than 1 mm. The above statements about granules apply correspondingly.

The curcuminoid formulations of the present invention show improved oral bioavailability compared to non-formulated crystalline curcuminoid(s). The oral bioavailability can be determined in experiments involving oral administration of the curcuminoid formulation of the invention (or an corresponding amount of non-formulated curcuminoid(s)) to a subject and measuring the level of the curcuminoid(s) in a biological sample obtained from the subject over time, wherein the biological sample may be derived from a body fluid, for example serum, plasma, whole blood, or cerebrospinal fluid, and/or a tissue, e.g. from brain, liver, kidney or heart. For analysis, the curcuminoid level in the examined body fluid or tissue may be plotted against time, and the area under the curve (AUC), for example the total area under the curve from t=0 (time of administration) to t=∞ (=AUC_{0-∞}), or the area under the curve within a defined period, e.g. from t=0 to t=6 h (AUC₀₋₆ₕ), may be calculated. In general, a higher AUC relative to the AUC obtained by administration of non-formulated crystalline curcuminoid(s) indicates an improved bioavailability. The absolute bioavailability may be calculated from the resulting AUC data as percentage based on the corresponding AUC data obtained from intravenous administration of curcuminoid(s).

In particular aspects of the invention, the amount of curcuminoid in the blood, determined as AUC₀₋₆ₕ, after a single oral administration of a dose of the curcuminoid formulation of the present invention corresponding to 20 mg of total curcuminoids to a human subject or an animal subject, preferably to a rat, is significantly higher than after oral administration of the same amount of non-formulated crystalline curcuminoids, preferably at least 2 times, at least 3 times, at least 4 times, at least 6 times, at least 8 times, at least 10 times, or at least 15 times, and, for example, up to 30 times higher.

As used herein, the amount of curcuminoid in the blood being “significantly higher” means a statistically significant increase of this parameter in subjects after oral administration of 20 mg curcuminoid comprised in the formulation of the invention compared to the control (20 mg non-formulated crystalline curcuminoids). A statistical test known in the art, such as ANOVA or Student's t-test, may be used to determine the significance of this difference, wherein the p-value is at least <0.1, <0.05, <0.01, <0.005, <0.001 or <0.0001.

### EXAMPLES

### Example 1

### Preparation and Analysis of Solid Dispersion Products for Curcuminoid Formulations

Apparatus:

twin-screw extruder ZSK 18 MEGAIab (Coperion) equipped with a gravimetric feeder and a strand die with a diameter of 3 mm

Screw configurations A and B as described below

For the experiment described below two alternative types of extruder shafts with different screw configurations were applied. Each type of shaft carried a number of processing elements disposed axially one behind the other over the total shaft length arranged in three sections. About on third of the shaft positioned farthest upstream was a feeding and conveying section followed by about a further third comprising several mixing sections connected by intermediate conveying sections. The mixing sections comprised kneading blocks which consisted of cam disks mutually offset at an angle in a peripheral direction. The about one third of the shaft positioned farthest downstream was a discharging section.

Screw configuration A comprised four mixing sections. Screw configuration B comprised three mixing sections and was more aggressive than screw configuration A due to the presence of back-pressure elements designed to intensify mixing and/or homogenization.

The extruder barrel was divided into five heating zones. Heating zones 1 and 2 covered the feeding and conveying section. The mixing sections and the discharging sections were located in heating zones 3 to 5.

Materials:

hydroxpropylmethylcellulose (HPMC 2910 5 mPa·s)

lecithin (Ultralec® F Lecithin)

isomalt (Isomalt PF)

curcuminoids (Curcumin C3 Complex, Sabinsa)

Procedure:

Four different powder premixes, #1, #2, #3 and #4, 1.5 kg each, were prepared by manually blending the ingredients shown in Table 1 in a PE bag. Samples of the premixes were fed separately to the twin-screw extruder. Melt extrusion was performed under the conditions summarized in Table 2. The samples were tested on residual crystalline curcuminoids after extrusion (Table 3). No crystallinity was detectable in the solid dispersion products resulting from processing of samples #1.1, #1.2, #2.1 and #2.2 having a curcuminoid load of 10% w/w. The solid dispersion products of sample #3.1-3 and #4.1-3 having a curcuminoid load of 20% w/w showed various levels of residual curcuminoid crystallinity related to and consistent with the applied process parameters.

TABLE 1
Composition of premixes

| Premix | Raw material | Amount [% w/w] | Amount [g] |
|---|---|---|---|
| #1 | HPMC | 60 | 900 |
| | Isomalt | 10 | 150 |
| | Lecithin | 20 | 300 |
| | Curcuminoids | 10 | 150 |
| #2 | HPMC | 75 | 1125 |
| | Isomalt | 5 | 75 |
| | Lecithin | 10 | 150 |
| | Curcuminoids | 10 | 150 |
| #3 | HPMC | 50 | 750 |
| | Isomalt | 10 | 150 |
| | Lecithin | 20 | 300 |
| | Curcuminoids | 20 | 300 |
| #4 | HPMC | 65 | 975 |
| | Isomalt | 5 | 75 |
| | Lecithin | 10 | 150 |
| | Curcuminoids | 20 | 300 |

TABLE 2
Parameters used for melt extrusion

| | Feed | | Screw | | Temperature profil | Melt | |
|---|---|---|---|---|---|---|---|
| | rate | Vacuum | speed | Torque | of heating zones 1-5 | temp. at | Screw |
| Sample | [kg/h] | [mbar] | [rpm] | [%] | [° C.] | die [° C.] | configuration |
| #1.1 | 0.75 | non | 200 | 12 | 50/100/130/140/140 | 142 | A |
| #1.2 | 0.75 | 500 | 150 | 11-12 | 50/100/130/140/140 | 144 | A |
| #2.1 | 0.75 | non | 200 | 14-15 | 50/100/130/140/150 | 155 | A |
| #2.2 | 0.75 | non | 150 | 33 | 50/100/130/140/150 | 158 | B |
| #3.1 | 0.75 | 540 | 150 | 21 | 50/100/120/130/140 | 145 | B |
| #3.2 | 1.1 | 540 | 150 | 30 | 50/100/120/130/140 | 144 | B |
| #3.3 | 0.75 | 560 | 125 | 29 | 50/90/110/120/130 | 134 | B |
| #4.1 | 0.75 | non | 125 | 32 | 50/90/110/120/130 | 137-139 | B |
| #4.2 | 0.75 | non | 124 | 52 | 50/90/100/110/120 | 122 | B |

TABLE 3
Product after melt extrusion

| | | | Curcuminoid crystallinity |
|---|---|---|---|
| Sample | Strand color | Comment | (mean of n = 2)* |
| #1.1 | brown | yellow | below detection limit |
| #1.2 | lighter brown | agglomerates, more | below detection limit |
| | | briddle | |
| #2.1 | brown | good homogentity | below detection limit |
| #2.2 | dark brown | yellow agglomerates | below detection limit |
| #3.1 | brown | yellow agglomerates | ~11% |
| #3.2 | yellow/brown | less agglomerates | ~20% |
| #3-3 | yellow/orange | less agglomerates | ~25% |
| #4.1 | light brown | smooth, homogen | ~11% |
| #4.2 | yellow/orange | smooth, homogen | ~28% |
| #4.3 | yellow | smooth, homogen | ~21% |

*% curcuminoid crystallinity based on the total amount of curcuminoid

### Example 2

### Bioavailability of Orally Administered Curcuminoid Formulations in Rats

Animals:

adult Sprague Dawley rats (14-16 weeks old, average body weight 260-280 g)

Animals were obtained from Harlan Laboratories, Netherlands. Rats were acclimatized to the study area conditions for 3 days before dosing. Animals were housed in polycarbonate cages and maintained in controlled environmental conditions with 12 hr light and dark cycles. The temperature and humidity of the room ranged between 21 to 24° C. and 50 to 70%, respectively. Animals were fed rat pellet food ad libitum except when fasted and were provided with fresh water. The animals were fasted for 12 h prior to dose administration.

Study Design:

Each dosing group consisted of 5 rats. Rats were implanted with cannula in the jugular vein for blood sampling. The surgical preparation was performed under anesthesia two days before dosing as per approved protocols. In a typical study, animals were orally dosed with test substance. Blood samples were taken prior to dosing and 15, 30, 60, 90, 120, 240 and 360 min after dosing. K2-EDTA was used as the anti-coagulant. Blood samples were collected from the jugular vein of cannulated rats in all cases. Plasma was collected by centrifugation and stored at −80° C. until bioanalysis. Quantitative bioanalysis of formulated curcuminoids in the plasma samples were processed using protein precipitation with acetonitrile and analyzed by LC-MS/MS.

Test Samples:

curcuminoid formulations: samples #1.1, #1.2, #2.1, #2.2, #3.1, #3.2, #3.3, #4.1 and #4.2, prepared as described in example 1, stored at 4° C. until use

control: non-formulated crystalline curcuminoids

Dosing of Test Samples:

On the day of dosing, dosing solutions were prepared by mixing the respective test sample with 0.25% carboxymethylcellulose in H₂O. Each animal was orally administered with ˜2.5 ml dosing solution comprising 20 mg curcuminoids.

Determination of Curcuminoids in Rat Plasma:

0.1 ml plasma was precipitated using 0.25 ml of acetonitrile containing warfarin (1000 μg/ml) as an internal standard. Precipitated protein was removed by centrifugation and supernatant fraction was analyzed by LC-MS/MS. Details of bioanalysis are given below:
Curcumin LLOQ: 30.23 ng/ml ULOQ: 3415 ng/ml
Desmethoxycurcumin LLOQ: 9.339 ng/ml ULOQ: 1055 ng/ml
Bis-desmethoxycurcumin LLOQ: 1.676 ng/ml ULOQ: 189.0 ng/ml
r² value: plasma: −0.9800
Instrumentation: API 4000 LC-MS/MS with 1.4.2 Analyst version
Mode: ESI Negative mode
Experiment Type: MRM
Ion source type & ionization mode: ESI

TABLE 4
Transition details in LC-MS/MS

| | MS/MS | Dwell | DP | EP | CE | CXP |
|---|---|---|---|---|---|---|
| Analyte | transition | (msec) | (Volts) | (Volts) | (Volts) | (volts) |
| Curcumin | 367/133.90 | 150 | −63 | 10 | −43 | −7 |
| Desmethoxy | 306.9/118.90 | 150 | −60 | 10 | −24 | −7 |
| curcumin | | | | | | |
| Bis-desmethoxy | 306.9/118.9 | 150 | −60 | 10 | −24 | −7 |
| curcumin | | | | | | |
| Curcumin | 543./134.0 | 150 | −63 | 10 | −43 | −7 |
| glucuronide | | | | | | |
| Desmethoxy | 513.0/118.9 | 150 | −58 | 10 | −24 | −7 |
| glucuronide | | | | | | |
| Bisdesmethoxy | 483.0/118.9 | 150 | −60 | 10 | −24 | −7 |
| glucuronide | | | | | | |
| ISTD | 307.2/161.2 | 150 | −85 | 10 | −43 | −9 |
| Warfarin | | | | | | |

| Interface heater | Ion source (GS1) | CAD | CUR | TEM | IS | GS2 |
|---|---|---|---|---|---|---|
| ON | 5 | 6 | 10 | 450 | 5500 | 45 |

HPLC Conditions
HPLC type: Shimadzu UFLC (XR)
Column: Agilent Eclipse XDB C18; 100×4.6 mm; 5μ.
Injection volume: 15 μl
Auto sampler & Column Temperature: Auto sampler 5° C.; Column temperature 40° C.
Mobile phase A: 0.1% formic acid
Mobile phase B: 0.1% formic acid in acetonitrile
Flow rate 1.0 ml/min with split at 80:20 (20% into mass)
Total sample analysis time: 10.0 min

Determination Of Curcuminoid Glucuronide Concentration in Plasma:

In this study, it was observed that curcuminoids were rapidly converted to conjugates of glucuronide in rat plasma. Since reference standards for curcuminoid glucuronides were not available, these conjugates were generated in vitro using rat liver S9 fraction and further purified. Curcuminoid glucuronides were quantified using the reported molecular extinction coefficient for curcumin (4.88×10⁴ M⁻¹ cm⁻¹ at 420 nm). (Our finding that purified curcumin glucuronide showed absorption maxima at 254 nm, similar to curcuminoids strongly suggests that glucuronide conjugation does not affect the spectral properties of the chromophore in the curcumin molecule.) Since the response in LC-MS/MS for curcumin and its glucuronide conjugate were not similar, a correction factor of 60 was applied to obtain approximate levels of curcuminoid glucuronides formed in rat plasma. (Said correction factor was determined as the ratio of the peak area counts obtained for similar amounts (−1.4 ng) of curcumin (peak area count: 613007) and its glucuronide conjugate (peak area count: 10103).)

Data Analysis and Determination of PK Parameters:

Data obtained from bioanalysis was analyzed using WinNonlin (V5.2.1) to determine various PK parameters such as Kₑₗ, t_{1/2}, Tₘₐₓ, Cₘₐₓ, AUC₀₋₆ₕ, and AUC_{0-∞}.

Results:

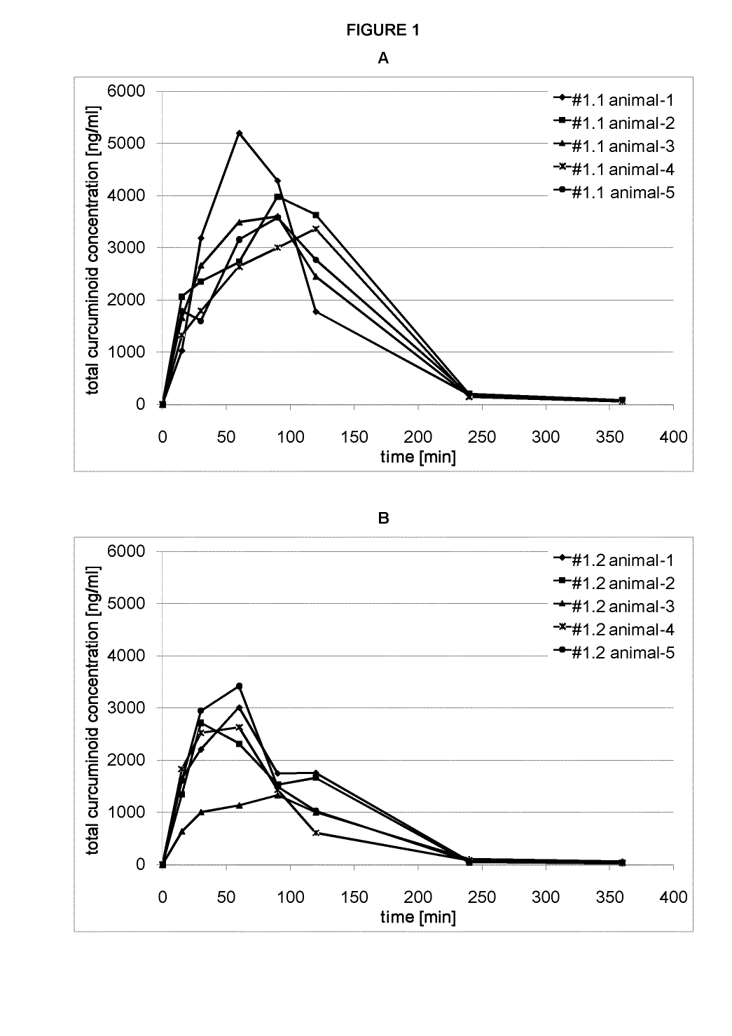

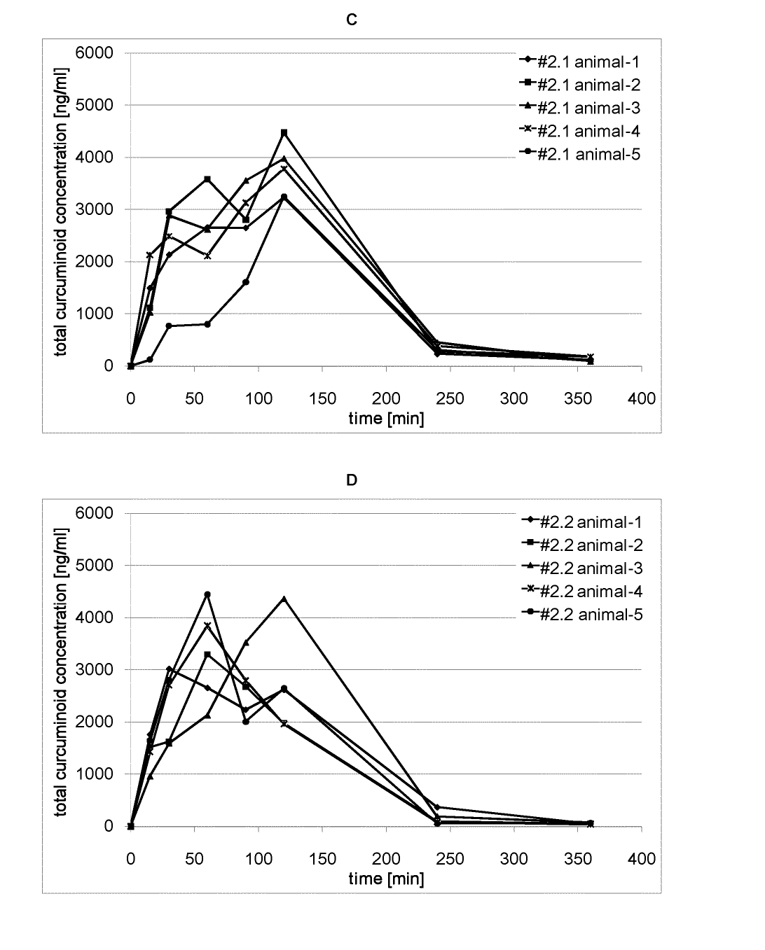

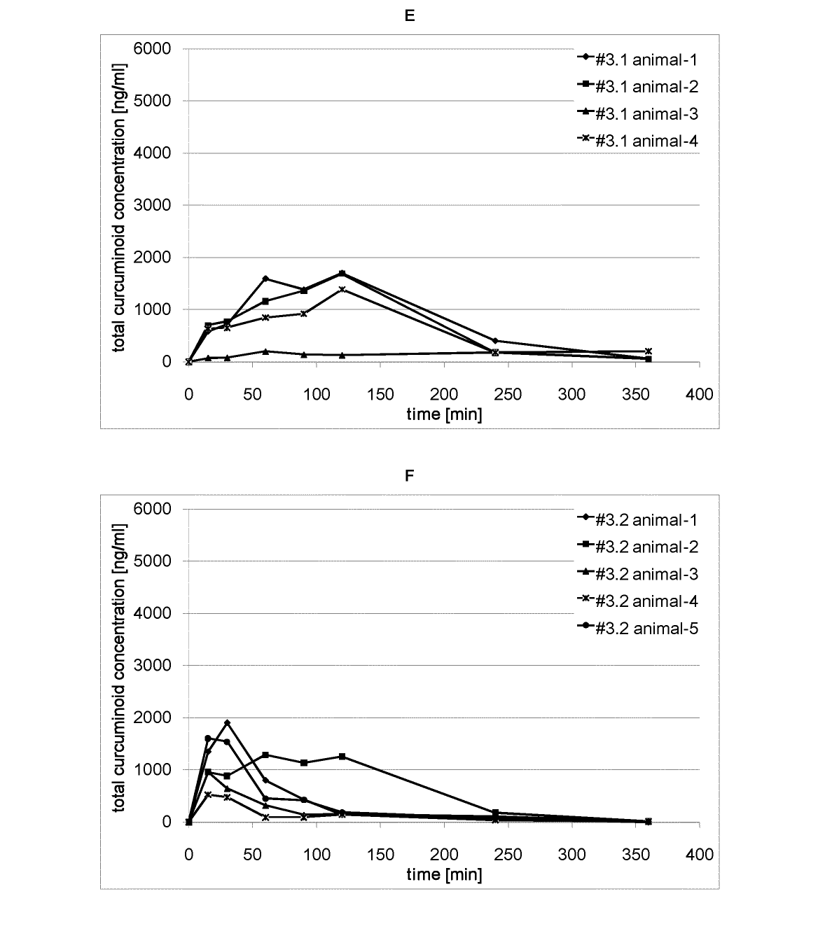

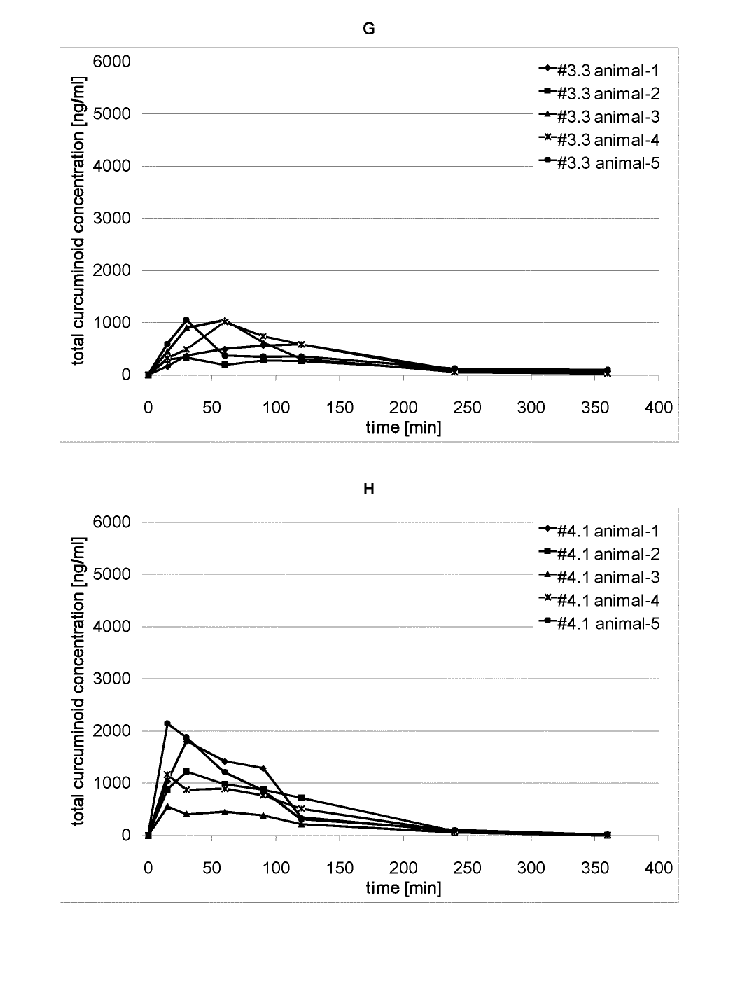

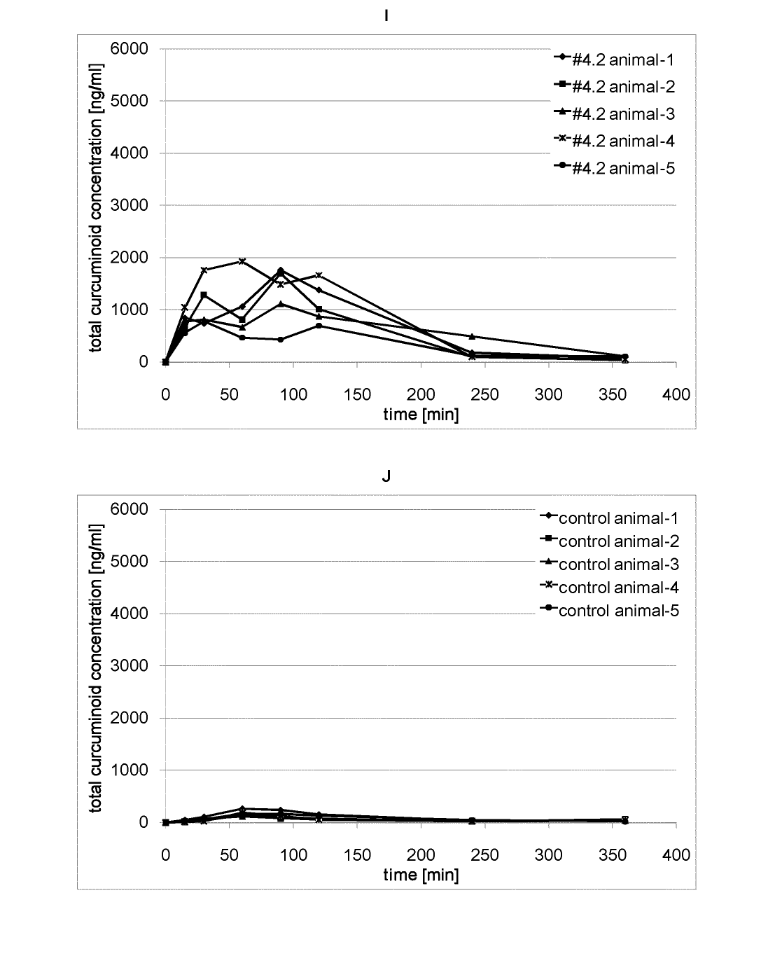

Calculated levels of curcuminoids (total curcuminoid glucuronides) in rat plasma measured from 0 min up to 360 min after administration of dosing solution and the respective pharmacokinetic parameters are shown in FIG. 1A-J and Table 5.

TABLE 5
Pharmacokinetic parameters for oral administration
of curcuminoid formulations to rats

| Test sample/ | | | | | Cₘₐₓ | AUC_{0-6 h} | AUC_{0-∞} |
|---|---|---|---|---|---|---|---|
| no. of rats | | Kₑₗ [1/h] | T_{1/2} [h] | Tₘₐₓ [h] | [ng/ml] | [h · ng/ml] | [h · ng/ml] |
| #1.1/5 | Mean | 0.77 | 0.90 | 1.50 | 3947 | 7401 | 7493 |
| | STDEV¹⁾ | 0.04 | 0.05 | 0.35 | 737 | 585 | 587 |
| #1.2/4²⁾ | Mean | 0.74 | 0.95 | 0.88 | 2945 | 4706 | 4751 |
| | STDEV | 0.10 | 0.13 | 0.22 | 310 | 384 | 381 |
| #2.1/5 | Mean | 0.72 | 0.97 | 2.00 | 3743 | 7357 | 7547 |
| | STDEV | 0.08 | 0.11 | 0.00 | 523 | 1635 | 1671 |
| #2.2/5 | Mean | 0.82 | 0.85 | 1.10 | 3795 | 6639 | 6702 |
| | STDEV | 0.07 | 0.08 | 0.55 | 633 | 637 | 642 |
| #3.1/3²⁾ | Mean | 0.64 | 1.24 | 2.00 | 1593 | 3501 | 3738 |
| | STDEV | 0.24 | 0.60 | 0.00 | 179 | 549 | 361 |
| #3.2/4²⁾ | Mean | 1.08 | 0.67 | 0.50 | 1439 | 1939 | 1945 |
| | STDEV | 0.26 | 0.19 | 0.35 | 406 | 851 | 850 |
| #3.3/5 | Mean | 0.46 | 1.66 | 1.00 | 809 | 1455 | 1597 |
| | STDEV | 0.15 | 0.61 | 0.61 | 332 | 339 | 373 |
| #4.1/5 | Mean | 1.20 | 0.63 | 0.35 | 1377 | 2149 | 2155 |
| | STDEV | 0.38 | 0.20 | 0.14 | 617 | 720 | 723 |
| #4.2/4²⁾ | Mean | 0.68 | 1.05 | 1.38 | 1624 | 3403 | 3502 |
| | STDEV | 0.11 | 0.18 | 0.25 | 355 | 531 | 521 |
| control/5 | Mean | 1.06 | 0.67 | 1.00 | 174 | 377 | 419 |
| | STDEV | 0.19 | 0.12 | 0.00 | 57 | 114 | 109 |

¹⁾STDEV = standard deviation
²⁾#1.2/animal-3, #3.1/animal-3, #3.2/animal-4, and #4.2/animal-5 were excluded from calculation of means and standard deviations.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. A curcuminoid formulation, comprising a melt-processed solid dispersion product comprising
a) one or more curcuminoids,
b) a nutritionally acceptable thermoplastic polymer, and
c) a phosphatide.

17. The curcuminoid formulation of claim 16, wherein the solid dispersion product additionally comprises a normally solid polyol.

18. The curcuminoid formulation of claim 16, wherein the curcuminoid(s) are present in an essentially non-crystalline state.

19. The curcuminoid formulation of claim 16, wherein the melt-processed solid dispersion product comprises
a) 0.1 to 50% by weight of the curcuminoid(s),
b) 20 to 95% by weight of the nutritionally acceptable thermoplastic polymer, and
c) 5 to 50% by weight of the phosphatide.

20. The curcuminoid formulation of claim 19, wherein the melt-processed solid dispersion product further comprises 0 to 50% by weight of a normally solid polyol.

21. The curcuminoid formulation of claim 16, wherein the nutritionally acceptable thermoplastic polymer is selected from cellulose derivatives.

22. The curcuminoid formulation of claim 16, wherein the nutritionally acceptable thermoplastic polymer is hydroxypropyl methyl cellulose.

23. The curcuminoid formulation of claim 20, wherein the normally solid polyol is a sugar alcohol.

24. The curcuminoid formulation of claim 20, wherein the normally solid polyol is isomalt.

25. The curcuminoid formulation of claim 16, wherein the phosphatide is lecithin.

26. The curcuminoid formulation of claim 16, wherein the curcuminoid formulation has a bioavailablity such that a single oral dose containing 20 mg of curcuminoids generates an increased curcuminoid blood concentration (determined as AUC₀₋₆ₕₒᵤᵣₛ) in a rat that is 2-30 times higher than that generated after oral administration of an equivalent dose of non-formulated crystalline curcuminoid.

27. A curcuminoid formulation according to claim 16, further comprising protein, carbohydrate and optionally fat.

28. A method of treating or preventing at least one of cancer, conditions involving an inflammatory reaction, neurological disorders, cardiovascular disease, pulmonary disease, the formation of cholesterol gallstones, and parasitic infestation, the method comprising administering an effective amount of a curcuminoid formulation comprising a melt-processed solid dispersion product comprising a) one or more curcuminoids, b) a nutritionally acceptable thermoplastic polymer, and c) a phosphatide to a subject in need thereof, wherein administration of the curcuminoid formulation results in treatment or preventing of the cancer, conditions involving an inflammatory reaction, neurological disorders, cardiovascular disease, pulmonary disease, the formation of cholesterol gallstones, or parasitic infestation.

29. A method according to claim 28, wherein the curcuminoid formulation has a bioavailablity such that a single oral dose containing 20 mg of curcuminoids generates an increased curcuminoid blood concentration (determined as AUC₀₋₆ₕₒᵤᵣₛ) in a rat that is 2-30 times higher than that generated after oral administration of an equivalent dose of non-formulated crystalline curcuminoid.

30. A method for producing a curcuminoid formulation comprising:
a) blending one or more curcuminoids, a nutritionally acceptable thermoplastic polymer, and a phosphatide;
b) heating the blend to obtain a homogeneous melt;
c) forcing the homogeneous melt through one or more nozzles to produce a forced melt; and
d) allowing the forced melt to solidify to obtain a solid dispersion product.

31. The method of claim 30, wherein b) is carried out in an extruder and the blend is subjected to a mixing action in a mixing section of the extruder.

32. The method of claim 30, wherein 0.1 to 50% by weight of the curcuminoid(s), 20 to 95% by weight of the nutritionally acceptable thermoplastic polymer, and 5 to 50% by weight of the phosphatide is utilized in a).

33. The method of claim 30, wherein the blending in a) further comprises a normally solid polyol.

34. The method of claim 30, wherein the nutritionally acceptable thermoplastic polymer is hydroxypropyl methyl cellulose and the phosphatide is lecithin.

35. The method of claim 33, wherein the nutritionally acceptable thermoplastic polymer is hydroxypropyl methyl cellulose, the phosphatide is lecithin, and the normally solid polyol is isomalt.
